(19) United States
(12) Patent Application Publication
(10) Pub. No.: US 2018/0127376 A1
(43) Pub. Date: May 10, 2018

(54) NOVEL CRYSTALLINE FORM OF ESLICARBAZEPINE

(71) Applicant: LUPIN LIMITED, Mumbai (IN)

(72) Inventors: Nandu Baban BHISE, District-Raisen (IN); Pravin Shamjibhai PATEL, Vadodara (IN); Hitendra Kanaiyalal MAHETA, Vadodara (IN); Daxesh Prakashbhai PATEL, Vadodara (IN); Harshad Tulsidas KHANVILKAR, Vadodara (IN); Priya Dipak RAKSHE, Vadodara (IN); Arpit Kiritbhai PATEL, Vadodara (IN)

(21) Appl. No.: 15/808,657

(22) Filed: Nov. 9, 2017

(30) Foreign Application Priority Data
Nov. 10, 2016 (IN) 201621038455

(51) Int. Cl. C07D 223/22

(52) U.S. Cl. CPC C07D 223/22; C07B 2200/13

(57) ABSTRACT

The present invention provides novel crystalline form L1 of eslicarbazepine characterized by diffraction peaks at 7.09, 10.03, 11.73, 14.12, 16.94, 18.03, 20.00, 23.20, 23.58, 23.76, 26.05, 26.52, 28.37, 29.90, 31.42±0.2 degree two theta in an X-ray diffraction pattern. The present invention further provides conversion of crystalline form L1 of eslicarbazepine to eslicarbazepine acetate.

### FIELD OF THE INVENTION

The present invention relates to novel crystalline form L1 of eslicarbazepine, process for its preparation and conversion to eslicarbazepine acetate.

### BACKGROUND OF THE INVENTION

Eslicarbazepine acetate (I) is a prodrug which is efficiently absorbed in the gastrointestinal tract and is metabolized to eslicarbazepine (II) by hydrolysis of the acetate group. Eslicarbazepine acetate (I) is indicated for the treatment of partial-onset seizure as monotherapy or adjunctive therapy under the trade name Aptiom®. The chemical name for eslicarbazepine acetate (I) is (S)-10-acetoxy-10,11-dihydro-5H-dibenz[b,f]axepine-5-carboxamide and eslicarbazepine (II) is (S)-10-hydroxy-10,11-dihydro-5H-dibenz[b,f]axepine-5-carboxamide. Eslicarbazepine acetate (I) and eslicarbazepine (II) has the following structural formula:

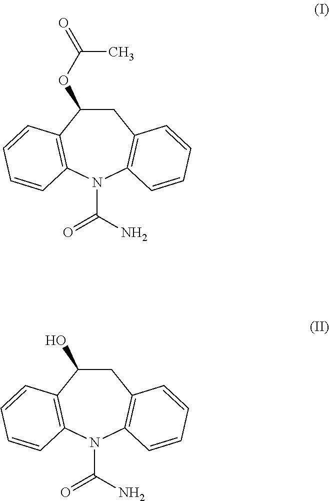 [SMILES: CC(=O)O[C@H]1Cc2ccccc2N(C(N)=O)c2ccccc21 ; NC(=O)N1c2ccccc2C[C@H](O)c2ccccc21]

The U.S. Pat. No. 5,753,646 describes eslicarbazepine acetate (I) and eslicarbazepine (II) and process for their preparation. The U.S. Pat. No. 7,119,197 provides process for preparation of optically pure eslicarbazepine (II). Another patent U.S. Pat. No. 9,346,760 provides process for preparation of eslicarbazepine (II) and eslicarbazepine acetate (I) and also provides polymorphic form of eslicarbazepine (II) namely Form J₁, J₂, J₃, J₄ and amorphous form.

The present invention provides novel crystalline form of eslicarbazepine (II) and its conversion to eslicarbazepine acetate (I).

### SUMMARY OF THE INVENTION

The present invention provides novel crystalline form L1 of eslicarbazepine characterized by diffraction peaks at 7.09, 10.03, 11.73, 14.12, 16.94, 18.03, 20.00, 23.20, 23.58, 23.76, 26.05, 26.52, 28.37, 29.90, 31.42±0.2 degree two theta in an X-ray diffraction pattern. The present invention further provides conversion of eslicarbazepine form L1 to eslicarbazepine acetate.

### DESCRIPTION OF DRAWINGS

FIG. 1—X-ray powder diffraction pattern of crystalline form L1 of eslicarbazepine.

FIG. 2—DSC thermogram of crystalline form L1 of eslicarbazepine.

### DETAILED DESCRIPTION OF THE INVENTION

The present invention relates to novel crystalline form L1 of eslicarbazepine and its conversion to eslicarbazepine acetate.

In one embodiment, the present invention provides crystalline form L1 of eslicarbazepine.

The crystalline form L1 of eslicarbazepine having characteristic diffraction peaks at 7.09, 10.03, 11.73, 14.12, 16.94, 18.03, 20.00, 23.20, 23.58, 23.76, 26.05, 26.52, 28.37, 29.90, 31.42±0.2 degree two theta in an X-ray diffraction pattern.

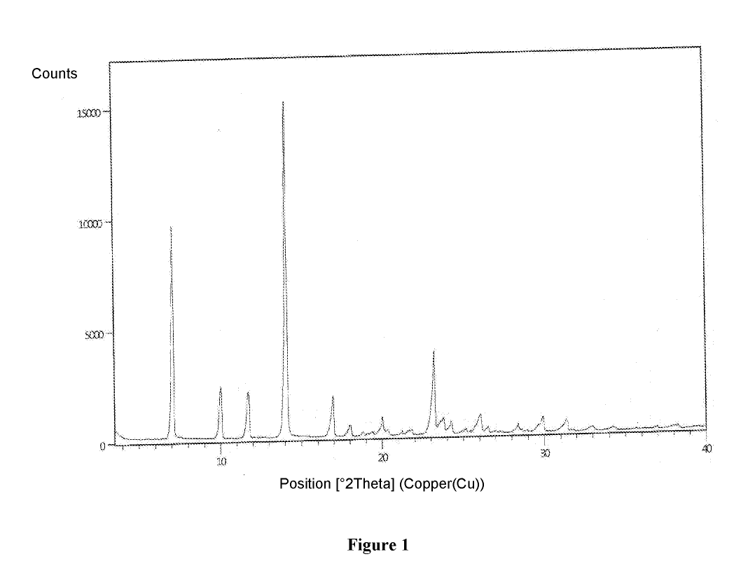

The crystalline form L1 of eslicarbazepine of the present invention is characterized by X-ray powder diffraction pattern as depicted in FIG. 1.

The crystalline form L1 of eslicarbazepine having characteristic infrared absorption at 3350, 3336, 3053, 3029, 2943, 2908, 1654, 1603, 1567, 1487, 1453, 1363, 1271, 1247, 1211, 1157, 1031, 985, 897, 882, 867, 777, 773, 651±2 cm⁻¹.

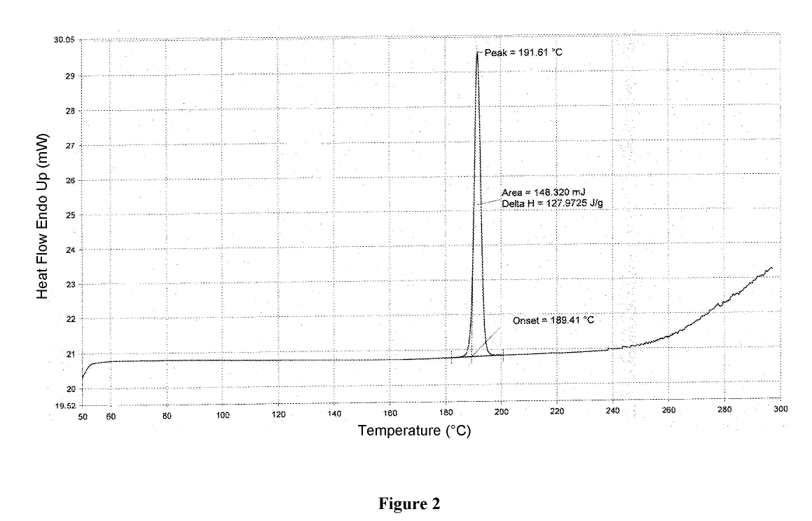

The crystalline form L1 of eslicarbazepine of the present invention having an endothermic peak in the range of 189 to 200° C. and peak temperature of about 191° C., in the differential scanning calorimetry (DSC). The crystalline form L1 of eslicarbazepine of the present invention is characterized by DSC thermogram as depicted in FIG. 2.

In yet another embodiment, the present invention provides a process for the preparation of crystalline form L1 of eslicarbazepine comprising the steps of:
a) dissolving eslicarbazepine in a solvent or mixture of solvents,
b) cooling the mixture,
c) isolating form L1 of eslicarbazepine.

Solvents can be selected from organic polar solvent or non-polar solvents, water or mixtures thereof. Polar solvent can be selected from alcohols like methanol, ethanol, butanol, propanol; nitriles like acetonitrile, propionitrile, butyronitrile; ethers like tetrahydrofuran, dioxane, dimethoxyethane; esters like ethyl acetate, ethyl acetoacetate, butyl acetate, propyl acetate; ketones like acetone, methyl ethyl ketone, methyl isobutyl ketone; other polar solvents like dimethylformamide, dimethyl sulfoxide, water and mixtures thereof. Non-polar solvents can be selected from hydrocarbon like hexane, cyclohexane, n-heptane, pentane, cyclopentane, toluene; chlorinated solvents like methylene chloride, ethylene chloride, chloroform, carbon tetrachloride and mixtures thereof.

Eslicarbazepine can be dissolved in solvent by heating the mixture of eslicarbazepine and solvent to a temperature of 25° C. to reflux temperature. The mixture can be cooled to 0-5° C. for a period ranging from 30 to 180 minutes and crystalline form L1 of eslicarbazepine can be isolated by techniques known in art like filtration, concentration, removal of solvent by evaporation, distillation, crash cooling, flash evaporation, drying on rotavapor.

Eslicarbazepine which is used for preparation of crystalline form L1 of the present invention can be prepared by methods as described in U.S. Pat. No. 7,119,197 or by preparations known in the literature.

In a further embodiment, the present invention provides conversion of crystalline form L1 of eslicarbazepine (II) to eslicarbazepine acetate (I).

Eslicarbazepine (II) crystalline form L1 can be reacted with acetylating agents selected from acetic anhydride, acetyl halide and the like; base selected from pyridine, dimethyl aminopyridine and the like. The reaction can be carried out optionally in presence of organic solvent selected from chlorinated solvents like methylene chloride, ethylene chloride, chloroform, carbon tetrachloride. Eslicarbazepine acetate (I) can be isolated from the reaction mixture by techniques known in the art.

The X-ray powder diffraction pattern was recorded at room temperature using PANalytical X'Pert PRO diffractogram with Cu Kα radiation (λ=1.54060 Å), running at 45 kV and 40 mA. The infrared absorption spectrum was obtained using a Perkin Elmer Precisely Spectrum 400 instrument using KBr pellet method. The DSC measurement was carried out on a Perkin Elmer in a closed pan with a heating rate of 10°/minute.

The present invention is further illustrated by the following representative examples and does not limit the scope of the invention.

### EXAMPLES

### 1. Process for Preparation of Crystalline Form L1 of Eslicarbazepine.

A mixture of eslicarbazepine (200 g) and isopropanol (1400 ml) was heated to 50° C., followed by distillation of 1000 ml of isopropanol, under vacuum. Water (1400 ml) was added to the mixture at 40-43° C. under stirring. The mixture was cooled to 0-5° C. over a period of 120 minutes. The solid was filtered and dried under vacuum. Yield: 185 g. Eslicarbazepine with X-ray powder diffraction pattern as depicted in FIG. 1 was obtained.

### 2. Process for Preparation of Eslicarbazepine Acetate.

A mixture of eslicarbazepine (150 g) of example 1, methylene dichloride (600 ml) and dimethyl aminopyridine (2.16 g) was stirred at 25-30° C. for 30 minutes. Acetic anhydride (77.63 g) was added to the reaction mixture over a period of 45 minutes and the reaction mixture was stirred for 60 minutes at 40° C. The mixture was cooled to 25° C. and the pH was adjusted to 7.5 to 8.5 by sodium bicarbonate solution. The organic layer was separated and washed with 0.35% hydrochloric acid and then with water, followed by carbon treatment. The organic layer was concentrated and isopropanol (900 ml) was added to the residue and further concentrated to remove traces of methylene dichloride. The mixture was heated to 80-85° C. and then stirred for 60 minutes. The mixture was cooled to 0-5° C. over a period of 120 minutes. The solid was filtered and dried under vacuum. Yield: 157.7 g.

1. A crystalline form L1 of eslicarbazepine having characteristic diffraction peaks at 7.09, 10.03, 11.73, 14.12, 16.94, 18.03, 20.00, 23.20, 23.58, 23.76, 26.05, 26.52, 28.37, 29.90, 31.42±0.2 degree two theta in an X-ray diffraction pattern.

2. The crystalline form L1 of eslicarbazepine of claim 1, characterized by X-ray powder diffraction pattern of FIG. 1.

3. The crystalline form L1 of eslicarbazepine of claim 1, characteristic by infrared absorption at 3350, 3336, 3053, 3029, 2943, 2908, 1654, 1603, 1567, 1487, 1453, 1363, 1271, 1247, 1211, 1157, 1031, 985, 897, 882, 867, 777, 773, 651±2 cm⁻¹.

4. The crystalline form L1 of eslicarbazepine of claim 1, characteristic by endothermic peak in the range of 189 to 200° C. and peak temperature of about 191° C., in the differential scanning calorimetry.

5. The crystalline form L1 of eslicarbazepine of claim 1, characterized by DSC thermogram of FIG. 2.

6. The crystalline form L1 of eslicarbazepine of claim 1, wherein crystalline form L1 of eslicarbazepine is converted to eslicarbazepine acetate.

7. A process for preparation of crystalline form L1 of eslicarbazepine of claim 1, comprising the steps of:
a) dissolving eslicarbazepine in a solvent or mixture of solvents,
b) cooling the mixture,
c) isolating form L1 of eslicarbazepine.

8. The process according to claim 7, wherein solvent is organic polar solvent, non-polar solvents or mixtures thereof.

9. The process according to claim 8, wherein organic polar solvent is an alcohol, nitrile, ether, ester, ketone, dimethylformamide, dimethyl sulfoxide, water or mixtures thereof.

10. The process according to claim 8, wherein organic non-polar solvent is hydrocarbon, chlorinated solvents or mixtures thereof.
